(19) United States
(12) Patent Application Publication
(10) Pub. No.: US 2009/0142320 A1
(43) Pub. Date: Jun. 4, 2009

(54) METHOD TO MINIMIZE IL-17 PRODUCTION DURING nTREG CELL EXPANSION

(75) Inventors: TingHua Cao, Malvern, PA (US); Steven Charles Eck, West Chester, PA (US); Li Li, Downingtown, PA (US)

Correspondence Address: HISCOCK & BARCLAY, LLP, 2000 HSBC PLAZA, 100 Chestnut Street, ROCHESTER, NY, 14604-2404, US

(73) Assignee: Therakos, Inc.

(21) Appl. No.: 12/364,691

(22) Filed: Feb. 3, 2009

Related U.S. Application Data
(63) Continuation-in-part of application No. 12/325,464, filed on Dec. 1, 2008.
(60) Provisional application No. 60/992,347, filed on Dec. 5, 2007.
(60) Provisional application No. 60/991,301, filed on Nov. 30, 2007.

(51) Int. Cl. A61K 35/12; C12N 5/02; A61P 37/06

(52) U.S. Cl. 424/93.71; 435/377

(57) ABSTRACT

Disclosed in this specification is a method to promote the growth of CD4+CD25Foxp3+ nTreg cells in a culture while minimizing the production of IL-17. The resulting cells are useful in the treatment of immune-related diseases.

### CROSS-REFERENCE TO RELATED APPLICATION

This application is a continuation-in-part of co-pending U.S. patent application Ser. No. 12/325,464 filed Dec. 1, 2008, which claims the benefit of U.S. provisional patent application Ser. No. 60/991,301, filed Nov. 30, 2007, and Ser. No. 60/992,347, filed Dec. 5, 2007, which applications are incorporated herein by reference in their entirety.

### FIELD OF THE INVENTION

This invention relates, in one embodiment, to a method for minimizing IL-17 production in cell populations during the expansion of nTreg cells. The resulting nTreg cells are particularly useful for treating immune-mediated diseases including, but not limited to autoimmunity, organ transplant rejection and graft versus host disease (GvHD).

### BACKGROUND OF THE INVENTION

T regulatory (Treg) cells are important in maintaining the homeostatic balance of the human immune system and immune tolerance. One of the most well studied types of Treg cells is the naturally occurring Treg (nTreg) cell (CD4+CD25+Foxp3+ cell). Defects in either the nTreg cells or in Foxp3 have been linked to unfavorable immune responses such as autoimmunity, allergic response, and organ rejection. Conversely, administrations of healthy CD4+CD25+Foxp3+ nTreg cells have demonstrated therapeutic effects in the treatment of a variety of animal disease models. Although the nTreg cells are a small fraction of the circulating lymphocyte pool it has been found that nTreg cells can be expanded ex vivo to provide clinically useful quantities of nTreg cells. The possibility therefore exists for using ex vivo expanded nTreg cells to regulate the immune response of a human being.

During the process, nTreg cells are withdrawn from peripheral blood mononuclear cells (PBMC) using magnetic bead-based methods. The enriched nTreg cells are activated with anti-CD3/CD28 coated beads in the presence of high concentrations (1000 U/ml) of human recombinant IL-2. Although the purified cells are enriched for nTreg using the bead-base methods, the resulting sample is not pure. Due to the lack of nTreg-specific surface markers, the sample almost always contains non-Treg cells that expressed similar cell surface markers (e.g. CD4 and CD25). After about three weeks of culture time, the nTreg cell populations underwent over a one hundred fold expansion while maintaining their phenotypic expressions (CD25+ Foxp3+). Careful culturing conditions are needed to prevent the non-nTreg cells from expanding faster than the nTreg cells and disturbing the overall composition of the sample. The overgrowth of non-Treg cells during nTreg expansion not only reduces the potency and effectiveness of the nTreg cell therapy, but also provides a potential source of pro-inflammatory T effector cells and cytokines. Thus there is a need to find strategies and compounds to suppress the activation and growth of non-Treg cells in the cultured population.

### SUMMARY OF THE INVENTION

Applicants have discovered that human IL17 was produced by a portion of the non-nTreg cells from ex vivo expanded human nTreg cell pool. Since the nTreg cells were well known for their immunosuppressive properties, it was surprising to discover the production of IL-17 in the expansion culture, which IL17 is known to promote immune mediated diseases.

IL-17 (JI17A) is a cytokine secreted mainly by activated CD4 and CD8 T cells. In vitro, IL-17 induces the production of several pro-inflammatory cytokines/chemokines (TNFa, IL-1b, IL-6, IL-8, GM-SCF, and MCP-1) from various cells types and also may play a role in neutrophil mediated inflammatory responses. In animal studies, it has been suggested that IL-17 may be involved in the pathogenesis of rheumatoid arthritis, osteoarthritis, asthma, inflammatory bowel disease, and multiple sclerosis. The production of IL-17 is regulated by other cytokine and co-stimulatory molecules. In mouse system, TGFβ, which is a key cytokine for the development and functions of certain types of Treg, is also the key differentiation cytokine for IL-17-producing T cells (Th17) in the presence of IL-6. Both IL-23 and IL-15 enhances, while IL-27 reduces, IL-17 production in mouse T cells. Certain co-stimulatory molecules also play a role in IL-17 production, ICOS up-regulate while OX40 down-regulate IL-17 production in T cells. Because of the potential pathological impact of IL-17 in several immune mediated inflammatory diseases, applicants believe it is desirable to minimize IL-17 production in cell populations that will be used to treat inflammatory diseases (for example, CD4+CD25+Foxp3+ nTreg cells).

Disclosed in this specification are several methods to minimize the production of IL-17 while expanding a cell population that includes CD4+CD25+Foxp3+ nTreg cells.

### BRIEF DESCRIPTION OF THE DRAWINGS

The present invention is disclosed with reference to the accompanying drawing, wherein:

FIG. 1 is a graphical depiction of the response of a cell culture to various additives.

The examples set out herein illustrate several embodiments of the invention but should not be construed as limiting the scope of the invention in any manner.

### DETAILED DESCRIPTION

Human natural Treg (nTreg) cells were purified from normal donor PBMC using the commercially available Miltenyi Treg kit with AutoMacs (available from Miltenyi). The resulting sample was enriched nTreg cells relative to the original sample. Similar results were obtained by depleting CD19+ cells and thereafter performing positive selection of CD25+ by AutoMacs using Miltenyi CD19 and CD25 beads. Typically, 50-70% of the enriched cells are Foxp3+ as assessed by intracellular Foxp3 staining and analyzed by flow cytometry. In some studies, nTreg was enriched through FACS sorting based on CD4+, CD25high and CD127low population. In this case, the Foxp3+ cells consisted of >90% of the CD4+CD25+ population.

The enriched nTreg cells were re-activated with anti-CD3/CD28 coated beads (Dynal) at a 1:3 cell to bead ratio in X vivo-15 media (Cambrex) that was supplemented with 10% pooled human serum (Cambrex) in the presence of 1000 (IU/ml) of human rIL-2 under 37° C. incubation. In three weeks time, nTreg cells experience over a one hundred fold expansion. About 50% of the expanded cells were Foxp3+ and the cultured cells exhibited potent inhibitory activities during in vitro functional assays. nTreg cells which had been cultured for three weeks were allowed to rest in IL-2-only media for two days and then re-stimulated with anti-CD3/CD28 antibodies. Cytokines in the supernatants were measured with Luminex. Cytokine profiles of the resulting cellular sample, as well as those from comparable CD25-T cells, were as follows:

Cytokine profile of cellular samples

| Cytokine | CD25− | | CD25+ | |
|---|---|---|---|---|
| IL-2 | 10,000-12,000 | pg/ml | None detected | |
| IL-10 | <10,000 | pg/ml | 50,000-60,000 | pg/ml |
| TNFa | 2500-3000 | pg/ml | <500 | pg/ml |
| IL-6 | <100 | pg/ml | 100-300 | pg/ml |
| GMCSF | ca. 50,000 | pg/ml | 10,000-20,000 | pg/ml |
| IL-17 | 10,000 | pg/ml | 1,000 | pg/ml |

Subsequent studied showed that a minor population of ex vivo expanded CD4+CD25+ T cells produce IL-17 and about 50% of the IL-17-producing cells are also Foxp3+. This data was determined as follows: A population of nTreg cells was purified from normal donor PBMC either using magnetic beads or through FACS sorting (CD4+CD25highCD127−) and the starting population of Foxp3+ cells was approximately 60%. Cells were activated with anti-CD3/CD28 in the presence of IL-2 for three weeks. The cells were then rested for two days and re-stimulated with PMA plus ionomycin for five hours and the IL-17 production was detected by intracellular staining for IL-17 and counter-stained for Foxp3.

Meanwhile, IL17 Intracellular staining was also performed on the enriched nTreg prior to expansion of the population. It was determined that IL17+ cells were present prior to expansion and the majority of the IL-17+ cells were Foxp3−. Additionally, the production of IL-17 from CD25− cells was also examined, but no detectable amount of IL-17 was found.

These results suggest that CD25+IL-17+(Foxp3+ or Foxp3−) T cells are present in the PBMC of normal donors. The Foxp3+IL17+ T cells in the three week nTreg cell samples could have come from the expansion of the minor population of the starting Foxp3+Il-17+ cells or were converted from the Foxp3-IL-17+ cells. Since there is a lack of unique cell surface markers to remove the IL-17+ cells from the nTreg culture, a practical strategy is to limit the expansion/conversion of the IL-17+ cells in the nTreg ex vivo culture through the manipulation of culture conditions.

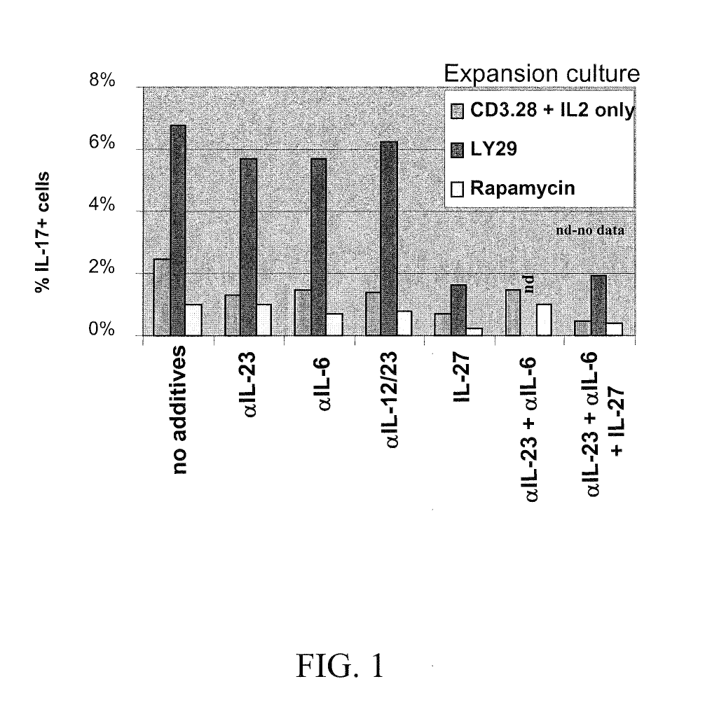

The production of IL-17 is known to be regulated by other cytokines including, for example, IL-6, IL-12, IL-23 and IL-27. The phrase “IL-17 inhibitor” refers to any compound capable of reducing the level of IL-17 expression, either directly or indirectly. Several cultures were treated with IL-17 antagonists. FIG. 1 shows the percentage of IL-17+ cells when various additives are used.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

What is claimed is:

1. A process for producing CD4+CD25+ nTreg cells comprising the steps of:
enriching CD4+CD25+ regulatory T cells in a sample thus producing enriched CD4+CD25+ regulatory T cells;
expanding the population of the enriched CD4+CD25+ regulatory T cells while treating the enriched cells with an IL-17 inhibitor.

2. The process as recited in claim 1, wherein the IL-17 inhibitor is an anti-IL-6 antibody.

3. The process as recited in claim 1, wherein the IL-17 inhibitor is an anti-IL-12 antibody.

4. The process as recited in claim 1, wherein the IL-17 inhibitor is an anti-IL-23 antibody.

5. The process as recited in claim 1, wherein the IL-17 inhibitor is IL-27.

6. The process as recited in claim 1, wherein the IL-17 inhibitor includes at least two compounds selected from the group consisting of an anti-IL-6 antibody, an anti-IL-12 antibody, an anti-IL-23 antibody, and IL-27, and combinations thereof.

7. A process for producing CD4+CD25+ nTreg cells comprising the step of expanding the population of the enriched CD4+CD25+ regulatory T cells while treating the sample with an IL-17 inhibitor.

8. A process for producing CD4+CD25+ nTreg cells comprising the steps of:
expanding the population of the enriched CD4+CD25+ regulatory T cells while treating the sample with an IL-17 inhibitor; and
administering a portion of the expanded CD4+CD25+ regulatory T cells to a human being to treat graft versus host disease.

9. The process as recited in claim 8, wherein the IL-17 inhibitor is an anti-IL-6 antibody.

10. The process as recited in claim 8, wherein the IL-17 inhibitor is an anti-IL-12 antibody.

11. The process as recited in claim 8, wherein the IL-17 inhibitor is an anti-IL-23 antibody.

12. The process as recited in claim 8, wherein the IL-17 inhibitor is IL-27.

13. The process as recited in claim 8, wherein the IL-17 inhibitor includes at least two compounds selected from the group consisting of an anti-IL-6 antibody, an anti-IL-12 antibody, an anti-IL-23 antibody, IL-27, and combinations thereof.
